(19) United States
(12) Patent Application Publication
(10) Pub. No.: US 2004/0091886 A1
(43) Pub. Date: May 13, 2004

(54) Method for generating recombinant polynucleotides

(76) Inventors: Jeffrey C. Moore, Scotch Plains, NJ (US); Jeffrey Bernstein, Westfield, NJ (US); James K. McCarthy, San Diego, CA (US)

Correspondence Address: MERCK AND CO INC, P O BOX 2000, RAHWAY, NJ, 070650907

(21) Appl. No.: 10/381,146

(22) PCT Filed: Sep. 17, 2001
(86) PCT No.: PCT/US01/29030

(51) Int. Cl. C12Q 1/68; C12P 19/34; C12N 9/22

(52) U.S. Cl. 435/6; 435/91.2; 435/199

(57) ABSTRACT

A novel method for producing recombinant polynucleotides in vitro is provided. This method entails the treatment of heteroduplex DNA sequences with a nuclease (preferably, DNase I) and a polymerase (preferably, DNA polymerase I), the enzymes primarily involved in nick translation. The results achieved using this process are superior to that achieved by previous in vivo recombination efforts utilizing specific DNA repair systems.

### FIELD OF THE INVENTION

This invention is directed to a novel method for producing recombinant polynucleotides in vitro.

### BACKGROUND OF THE INVENTION

The process of recombination is of incredible interest because of its ability to generate novel sequences possessing improved or desired functions. Naturally, this is an indiscriminate process. This has changed, however, with the design of procedures capable of achieving these same goals in a more deliberate and crafted manner. Procedures mimicking natural recombination events are now exploited to specifically generate and select for proteins or sequences particular to certain needs. Many methods exist whereby one of skill in the art can carry out these processes.

The difficulty is that many of these methods require a detailed understanding of the sequence and structure/function relationship of a particular protein of interest and, thus, are very time-consuming and information-intensive; see, e.g., Shao and Arnold, 1996 Curr. Opin. Struct. Biol. 6:513-518. Other methods are available that circumvent this time and energy expense, however, they have an expense of their own in, for example, requiring numerous cycles of selection and mutation in the pursuit of a sequence possessing the desired beneficial mutations (see, e.g., Kuchner and Arnold, 1997 Trends in Biotech. 15:523-530) or repeated PCR cycles in generating recombinant sequences that might be of interest (see, e.g., U.S. Pat. No. 5,605,793).

Heteroduplex molecules have been employed in the art as alternative substrates to the typical perfectly complementary (homoduplexed) double-stranded nucleic acid molecule and have been found to effect more directed recombination efforts, avoiding the very information- and/or labor-intensive methods available in the art; see, e.g., Lu et al., 1983 Proc. Natl. Acad. Sci. USA 80:4639-4643. Heteroduplex molecules in these processes can be created by a number of methods. Most often, the heteroduplex molecules are created by heating and annealing of the initial (parental) homoduplex nucleic acid molecule substrates.

Repair of these heteroduplex intermediate molecules has been employed in methods specifically designed to generate recombinant, novel sequences; see e.g., WO 99/29902; Volkov et al., 1999 Nucleic Acids Res. 27(18):e18 i-vi. The procedure disclosed in WO 99/29902, specifically, involves preparing a heteroduplex molecule in vitro from double-stranded nucleic acid molecules differing in sequence composition (referred to herein as variant homoduplex parental sequences or variant homoduplex molecules). This heteroduplex molecule is then introduced into a bacterial cell (E. coli) repair system where repair of the mismatched regions is effected resulting in a unique double stranded nucleic acid molecule (or homoduplex) different from the double-stranded nucleic acid starting material.

Repair of heteroduplex molecules in the E. coli bacterial cell is known in the art to be carried out in vivo through the mut HLS system; see e.g., Lu et al., supra; WO 99/29902; Volkov et al., 1999 Nucleic Acids Res. 27(18):i-vi. Mut HLS repair in vitro has been described in the art; see, e.g., Fang et al., 1997 J. Biol. Chem. 272(36):22714-22720.

It would be desirable to identify alternative systems capable of generating novel polynucleotides via the repair of mismatched heteroduplex molecules, particularly one that repairs heteroduplex molecules with a greater consistency and efficiency than the bacterial Mut HLS system. More desirable is such a process capable of effecting this repair in vitro such that the repair mechanism can be isolated, more closely analyzed, and optimized.

### SUMMARY OF THE INVENTION

The present invention relates to a novel method for producing recombinant polynucleotides in vitro wherein heteroduplex molecules are contacted with a mixture comprising a heteroduplex repair system consisting essentially of a polymerase, preferably, in the presence of a nuclease. The results achieved using this process are superior to that achieved by previous in vivo recombination efforts utilizing specific DNA repair systems.

### BRIEF DESCRIPTION OF THE DRAWINGS

FIG. 1 shows the nucleic acid sequences of wild-type lacZα (SEQ ID NO:5) and two constructed mutants of lacZα (M1 and M2; SEQ ID Nos: 1 and 2, respectively), aligned. Both M1 and M2 mutants contain four stop codons, two in-frame and two out of the reading frame. The M1 variant contains a 27 base mutation region starting at base 50 (from the ATG start) on the Lac Zα gene, including a 3 base deletion and 16 mismatched bases. The M2 variant contains a 22 base mutation region with 9 mismatched bases and a 3 base insertion starting at base 156 on the gene. The distance between the last in-frame stop codon on the M1 variant and the first in-frame stop codon on the M2 variant is 81 bases.

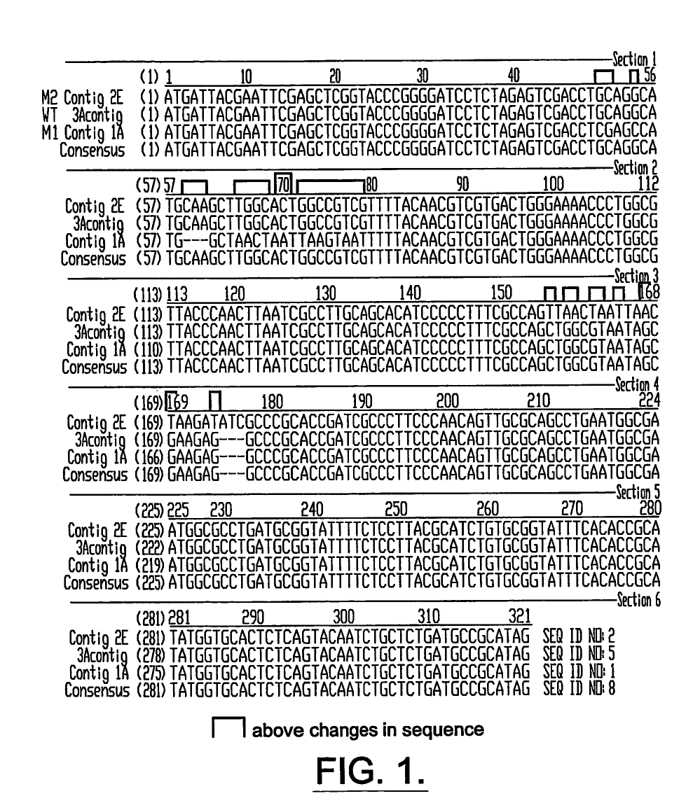

FIG. 2 illustrates a gel showing the DNA after the nick translation reaction (both uncut and cut with restriction enzymes before cloning) and the control after having been digested with restriction enzymes. From left, the lanes are as follows: 5 μL 1 KB Ladder; 2 μL digested ssDNA heteroduplex control, 2 μL digested dsDNA heteroduplex control; 2 μL digested ssDNA heteroduplex after nick translation; 2 μL digested dsDNA heteroduplex after nick translation with ligase; 2 μL digested dsDNA heteroduplex after nick translation; 3.51 μL Lac Zα insert DNA; 3.5 μL ssDNA heteroduplex after nick translation; 3.5 μL dsDNA heteroduplex after nick translation with ligase; and 3.5 μL dsDNA heteroduplex after nick translation.

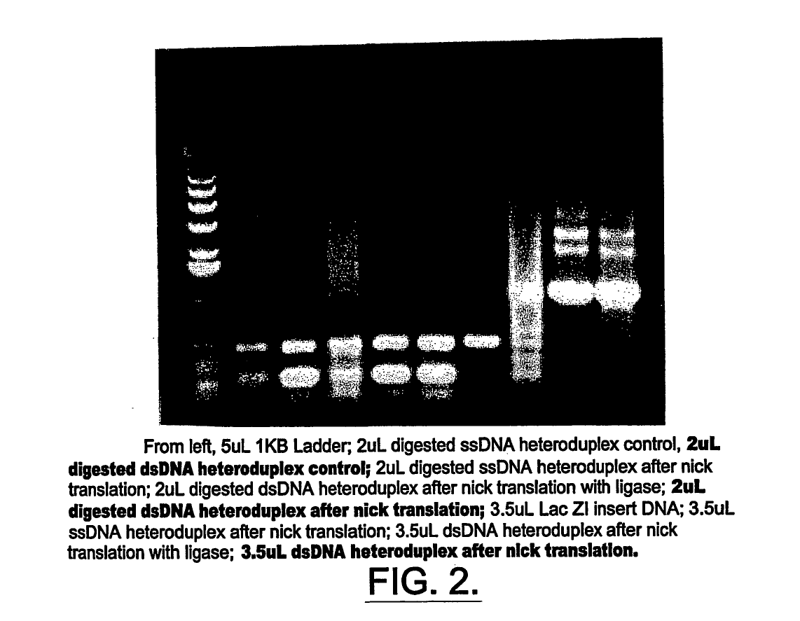

FIG. 3 shows the nucleic acid sequences of wild-type lacZα (SEQ ID NO:5) and two additional constructed mutants of lacZα (M3 and M4; SEQ D Nos: 3 and 4, respectively), aligned. Mutants M3 and M4 contain four stop codons, two in-frame and two out of the reading frame. Neither mutant has any insertions or deletions of bases relative to the wildtype Lac Zα gene. M3 contains a 14 base mutation region with 12 mismatched bases relative to wildtype while M4 has a 16 base mutation region and 8 mismatched bases. There are also 81 bases between the end of the last stop codon in M3 and the beginning of the first stop codon in M4.

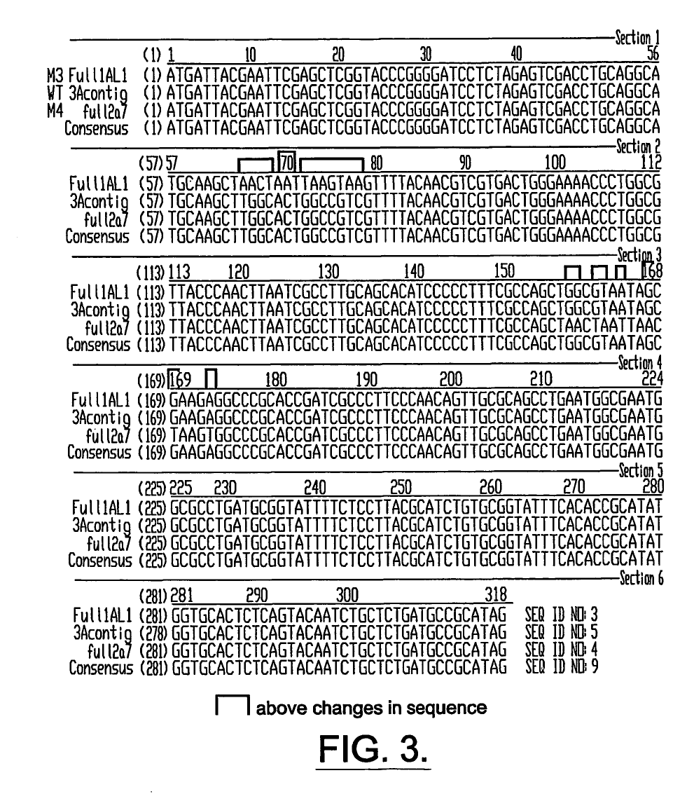

FIG. 4 illustrates, diagrammatically, the formation of heteroduplex molecules with mutants M1 and M2.

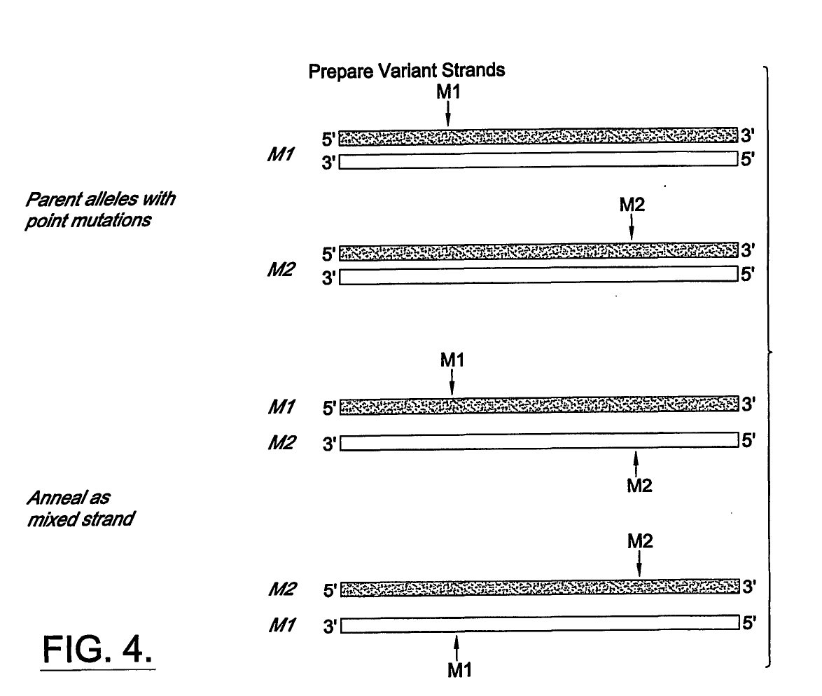

FIG. 5 shows a protocol for nicking the heteroduplex molecules of FIG. 4.

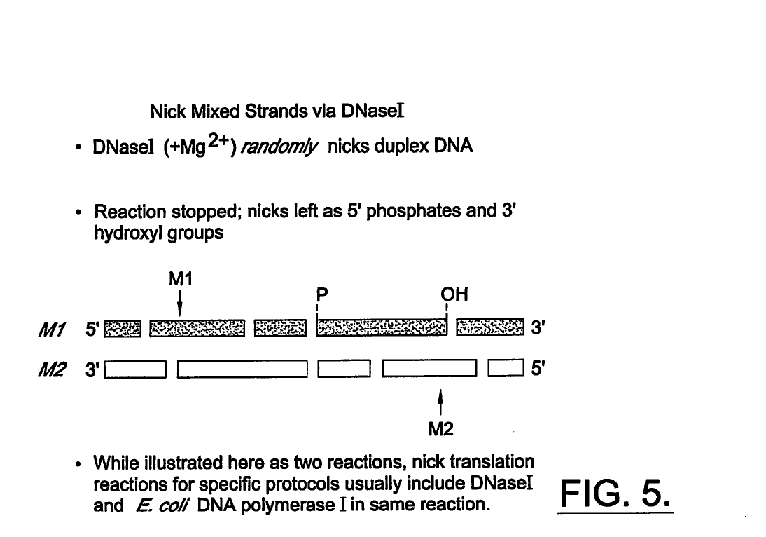

FIG. 6 shows the activity, diagrammatically, of DNA Pol I on the nicked molecule of FIG. 5.

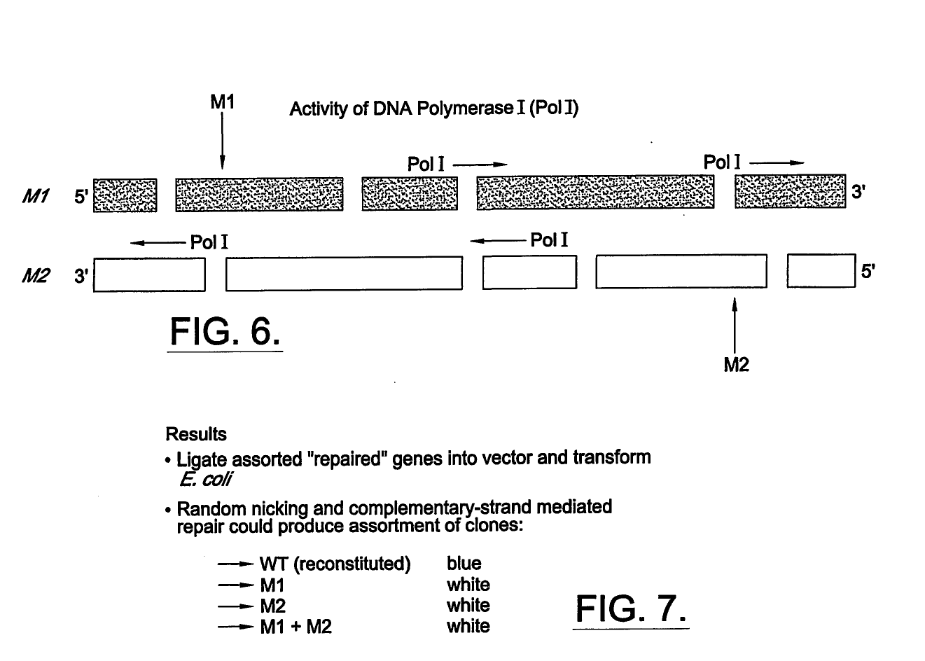

FIG. 7 shows the expected results using the blue/white screening assay discussed below.

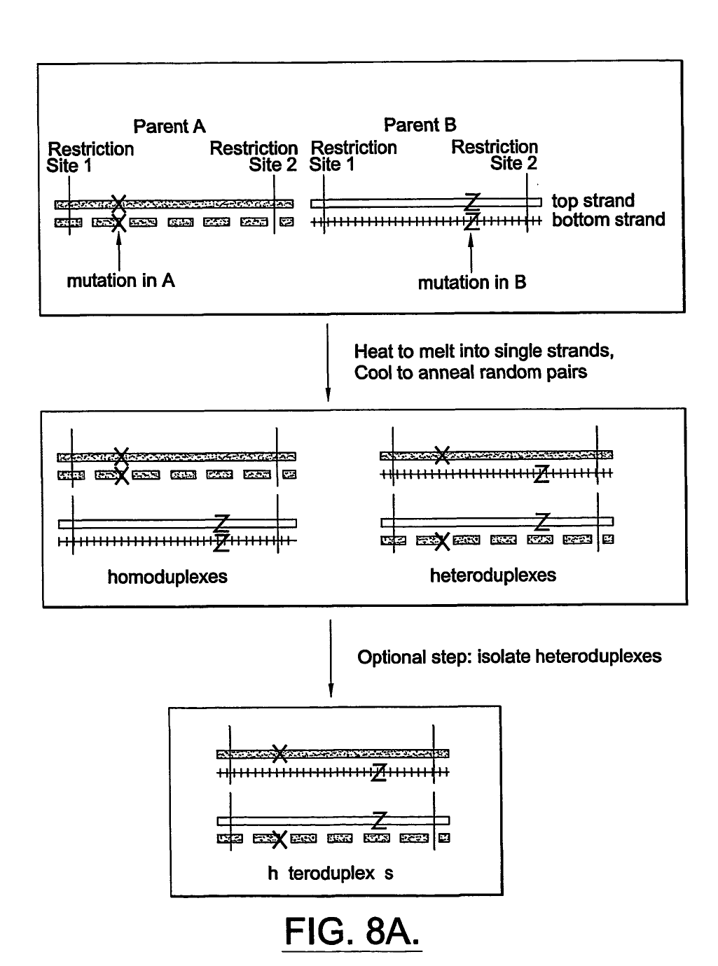

FIGS. 8A and 8B illustrate a general reaction carried out using the disclosed methods.

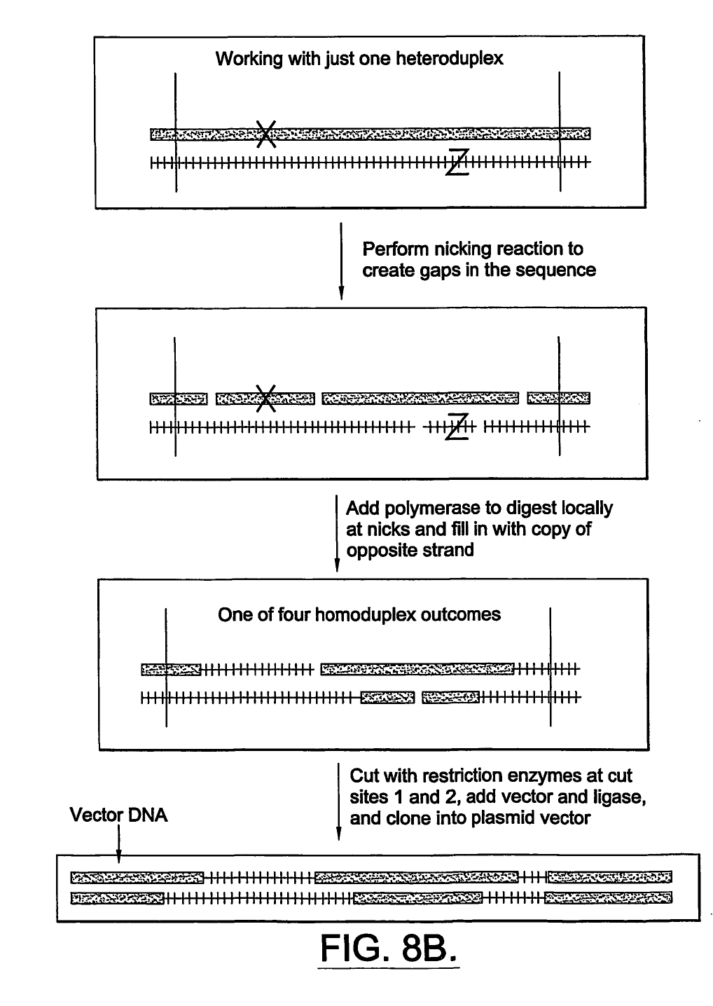

### DETAILED DESCRIPTION OF THE INVENTION

As used throughout the specification and claims, the following definitions apply:

“recombinant polynucleotides”, “recombinant sequences”, etc., refers to sequences, derived from recombination efforts disclosed herein, that differ in sequence composition from the original parental sequences (the variant homologous parental sequences, see below).

“recombination” refers to any process whereby a chimeric sequence is generated from the parental sequences or sequences derived therefrom.

“novel” or “chimeric” with respect to a particular polynucleotide refers to a sequence that differs in sequence composition from the variant homologous parental sequences (defined below) employed in a particular reaction.

“variant homologous parental sequences” refers to sequences that differ in sequence composition yet possess a degree of homology sufficient to allow hybridization of complementary DNA strands of the parental sequences. One of ordinary skill in the art is fully aware that hybridization conditions will vary based on the level of homology; see, e.g., Stemmer et al., 1994 Proc. Natl. Acad. Sci USA 91:10747-10751, wherein conditions were modified to accommodate a very low effective annealing temperature in order to generate chimeras from a human and a murine IL-1β gene possessing areas of sequence identity of on average only 4.1 bases long.

“conditions which promote heteroduplex formation” refers to any conditions that allow for heteroduplex formation between complementary strands of the parental homologous sequences.

“mixture” refers to any combination of reagents (e.g., enzymes, buffers, etc.) or materials employed within the instant invention.

“heteroduplex repair system” refers to the enzymes or enzyme used to effect recombination of the heteroduplexes by means of resolving mismatches between the two variant strands of the heteroduplex molecule in order to bring about the formation of recombinant polynucleotides.

“nuclease” refers to any protein capable of hydrolyzing a peptide bond within a nucleic acid sequence.

“polymerase” refers to any protein capable of catalyzing the addition of a nucleotide to the 3′ end of a nucleic acid molecule chain.

“Pol I” refers to DNA polymerase I.

“heteroduplex” refers to a double-stranded nucleic acid molecule wherein the composite strands are not 100% complementary. Preferably, they are substantially complementary (greater than 50%, more preferably, greater than 70%, even more preferably, greater than 80% and most preferably, greater than 90%). It is this molecule that is subject to the polymerase preferably with the nuclease. It is important that this be distinguished from that employed in Stemmer (U.S. Pat. No. 5,605,793). Therein the key intermediate was not a heteroduplex molecule as above described but, rather, a molecule wherein a particular strand of said molecule is derived from numerous fragments of the parental sequences.

“thermal melting point” or “Tm” refers to the temperature at which approximately 50% ±2% of a set of complementary probes hybridize to a target sequence at equilibrium under defined ionic strength, pH and nucleic acid concentration.

In accordance with the instant invention, Applicants have discovered a novel process wherein recombinant polynucleotides can be produced in vitro using a process employing a polymerase and, preferably, a nuclease. These enzymes have been found to resolve mismatches between variant heteroduplex strands and, in the process, generate novel sequences, i.e., sequences differing in sequence composition from the variant homologous parental sequences used in a particular reaction.

The polymerase, preferably in combination with the nuclease, is the main effector of the heteroduplex repair system. The repair system, however, can also comprise any other enzymes found to contribute to the heteroduplex repair process, so long as the actual recombination effects (i.e., generation of recombinant polynucleotides via resolution of mismatched nucleotides within the heteroduplex) is a direct result of the activity of the polymerase (in the case of a nicked heteroduplex molecule), or the polymerase and the nuclease.

A means of producing recombinant polynucleotide sequences from heteroduplex DNA which specifically employs and relies on nick translation enzymes (particularly, DNaseI, an endonuclease, and Pol I, a polymerase) or, alternatively, solely a polymerase (wherein the heteroduplex molecule has already been nicked) is new. The predominant method of use in the art for generating recombinant sequences from a heteroduplex has been a system employing the mutH, mutL and mutS enzymes of E. coli; e.g., see WO 99/29902.

The instant invention, therefore, relates to a method for producing recombinant polynucleotides, which comprises providing variant homologous parental sequences; incubating said sequences under conditions which promote heteroduplex formation; contacting said heteroduplexes with a mixture comprising a heteroduplex repair system consisting essentially of a nuclease (preferably, an endonuclease) and a polymerase; and identifying the recombinant polynucleotides produced. Alternatively, and additionally, the instant invention relates to a method for producing recombinant polynucleotides, which comprises providing variant homologous parental sequences; incubating said sequences under conditions which promote heteroduplex formation wherein the formed heteroduplexes are nicked; contacting said heteroduplexes with a mixture comprising a heteroduplex repair system consisting essentially of a polymerase; and identifying the recombinant polynucleotides produced. Additional methods provide a means of repairing mismatched nucleic acid molecules in vitro which comprises contacting the mismatched molecule with a mixture consisting essentially of a polymerase (wherein the mismatched molecule is nicked) or a polymerase and a nuclease.

The essence of the invention is that the reaction of nick translation is exploited in vitro to resolve mismatches between heteroduplex DNA derived from variant homologous parental sequences. Nick translation is known in the art primarily as a means of removing RNA primers or, alternatively, for producing uniformly radioactive DNA of high specific activity, and is thereby noted for the preparation of sequence-specific probes, for genomic DNA blots and for RNA blots. The concept of using the nick translation machinery in the generation of recombinant sequences from mismatched heteroduplex molecules is novel. Applicants were the first to adopt this very specific reaction to a very useful purpose—that of generating unique, potentially more desirable, sequences; sequences which perhaps encode proteins of improved properties.

Throughout the disclosure, it is to be noted that elements other than the nuclease and the polymerase (e.g., buffer materials, dNTPs, H₂O, etc.) can be added to the heteroduplex repair system to facilitate a specific reaction, hence the term mixture within the claim. This does not deny, however, that the repair mechanism is carried out as a direct result of the actions of the polymerase (in the event of a nicked heteroduplex) or the polymerase and the nuclease. Accordingly, the mixture must specifically comprise the heteroduplex repair system discussed above (mainly, the polymerase or polymerase/nuclease combination) but can add other reagents to impact the repair process in a desired manner.

The disclosed method was utilized in efforts to shuffle genetic material amongst two non-functional mutants of Lac Zα. Briefly, blue/white screening was conducted in the presence of X-Gal. Any recombination between the two mutant Lac Zα genes which resulted in a wildtype LacZα was identified by the release of an indigo blue product. Release of the indigo blue product was dependent on cleavage of the X-Gal by β-galactosidase that is encoded by wildtype Lac Zα. Non-functional mutant colonies appear white when expressed in the presence of X-Gal. By contrast, a successful experiment (where both mutations are removed from the heteroduplex DNA) reverts effected colonies back to a blue wildtype phenotype in the presence of X-Gal. Reversion to the wildtype as tested herein is one manner in which to readily detect and report specific recombination events; see, e.g., Stemmer (U.S. Pat. No. 5,605,793).

The results achieved employing the instant process were as follows: the nick translation reaction resulted in 5 blue colonies out of 524 colonies, for a 0.95% reversion frequency; while a heteroduplex control resulted in 7 blue colonies out of 1,032 colonies, for a 0.54% reversion frequency (roughly half as efficient as the nick-translated DNA). Similar experiments were run, finding a 0.8% reversion frequency (16/1,911) for the nick-translation-treated heteroduplex molecules, and a 0.2% reversion frequency (2/1,011) for the heteroduplex control group (roughly ¼ as efficient as the nick-translated version). The heteroduplex control employed in the examples was not subjected to nick translation processes but, simply, digested with restriction enzymes, ligated into a vector and transformed into E. coli cells, thus following the protocol of the nick-translated heteroduplex molecules all except for contact with the heteroduplex repair system (mainly, the polymerase or polymerase/nuclease combination). Thus, the mismatched heteroduplex control was subject only to cellular (E. coli) repair processes and, therefore, epitomized that employed in Arnold et al., WO 99/29902.

The recombinant polynucleotides of the instant invention are generated from heteroduplex molecules derived from variant homologous parental sequences. Variant homologous parental sequences of use in the instant invention are any sequences that differ in sequence composition (by at least one nucleotide) yet possess a degree of homology sufficient to allow hybridization of complementary sequences. Variant homologous parental sequences can be mutant forms or different alleles of the same sequence, they can encode the same protein but be present within different organisms or, alternatively, they can be of two or more different genes with a degree of homology sufficient for hybridization. The variant homologous parental sequences can further be present on vector DNA, examples of which are plasmids, cosmids, BACs (bacterial artificial chromosomes) and YACs (yeast artificial chromosomes). Preferably, the variant homologous parental sequences are from 50 to 150,000 basepairs, more preferably, 50 to 10,000, and most preferably, from 300 to 2000 basepairs. The sequences can also vary in length with respect to each other. Preferably, the sequences vary in length from about 50 to 5,000 basepairs. Most preferably, the sequences vary in length from 0-2,000 basepairs. The true goal here is sufficient hybridization in heteroduplex form in order to allow for a polymerase to act, preferably, in concert with a nuclease, to generate unique DNA from the heteroduplex.

As one of skill in the art will appreciate, hybridization conditions are generally sequence-dependent and vary according to the desired reaction. Preferably, hybridization conditions are determined based on the sequence and are roughly 25° C. lower than the thermal melting point (Tm) for the sequence of interest at a defined ionic strength and pH; all of which is readily determined by one skilled in the art. One of ordinary skill in the art will further appreciate that the annealing temperature can be varied in order to accommodate heteroduplex formation between variant sequences; see, e.g., Stemmer et al., supra, wherein conditions were modified to accommodate a very low effective annealing temperature in order to generate chimeras from a human and a murine IL-1β gene possessing areas of sequence identity of on average only 4.1 bases long.

The variant homologous sequences can exist in double-stranded or single-stranded form. Furthermore, they can be DNA (e.g., PCR product, genomic or cDNA) or RNA or any analog thereof. In the event that RNA is used, the RNA is transcribed to cDNA prior to heteroduplex formation for annealing with other DNA or cDNA products.

Most preferably, the sequences incorporated into the reaction possess a high degree of homology. Preferably, the sequences are at least 50% homologous, more preferably, at least 70% homologous and, most preferably, at least 90% homologous. Particularly preferred embodiments include variant homologous sequences with at least 99% sequence identity. Preferably, at least two sequences which differ in at least two basepair positions will be present in order to allow for the generation of a unique sequence from a recombination event.

The instant invention was demonstrated with a system wherein the variant homologous parental sequences were derived from the Lac Zα gene, particularly the mutants M1, M2, M3 and M4 (SEQ ID Nos: 1-4, respectively) of Lac Zα. This system is employed in the art (see U.S. Pat. No. 5,605,793) as an efficient, reliable means to demonstrate the viability of a particular gene shuffling method. As one of ordinary skill in the art will appreciate, though, this is not meant to impact its applicability to any of a number of “variant homologous sequences” as defined in the instant specification. It is easily understood that this system is amenable to use with any number of sequences that differ in sequence composition yet possess a degree of homology sufficient to allow hybridization of complementary DNA strands of different parental sequences.

Variant homologous parental sequences in accordance with the instant invention can be generated in any number of ways or they can be natural variants. Some preferred methods of producing variants are error-prone PCR, cassette mutagenesis, UV mutagenesis, site-directed mutagenesis, chemical mutagenesis, and in vivo mutagenesis; all of which are known in the art. One of skill in the art can appreciate, though, that any means capable of producing variant homologous sequences as defined in the instant disclosure is suitable for use in the instant invention.

Once the sequences are chosen and available, the sequences are placed under conditions in order to allow heteroduplex molecules to form from the variant parental sequences. A number of means exist in the art to generate heteroduplex DNA from homologous parental sequences; all of which can be used in the instant invention. The determination of conditions suitable for the heteroduplex formation for a particular set of sequences is, further, well within the realm of skill of one of ordinary skill in the art.

Preferred embodiments generate heteroduplex DNA by the heating and annealing of variant homologous sequences. More preferably, two homologous PCR products (PCRed for amplification purposes) containing desired mutations in appropriate buffer are heated and annealed together. PCR, in this instance, is used for the purpose of amplifying the initial substrates, the variant homologous parental sequences. It is not a necessary step of the actual recombination process. The instant process does not mandate the use of primers, nor does it depend on at least three rounds of PCR as does the method of Stemmer (U.S. Pat. No. 5,605,793).

Another means of producing heteroduplex DNA involves amplifying single-stranded plasmid DNA by use of M13-derived vectors and helper phage. In this embodiment, two complimentary single-stranded plasmids containing two homologous genes amplified in this manner are joined to create heteroduplex DNA and the nick translation reaction is run using entire plasmid.

Another preferred method of producing heteroduplex DNA involves running separate single strand PCR reactions for each of the homologous genes. In this manner, a low homoduplex background is produced. One reaction is run with only the forward primer using a parental sequence PCR product as template and the other reaction is run with the reverse primer and a “variant homologous parental sequence” (see definitions) PCR template. The forward and reverse single-stranded PCR products containing the two homologous parental sequences are then joined to create heteroduplex DNA. This heteroduplex DNA is then subjected to a nick translation reaction. The resultant product is then cut and cloned into a suitable plasmid expression vector.

In an alternate preferred embodiment, one mutant plasmid is digested with a unique restriction enzyme upstream of the gene (e.g., EcoRI) and the other mutant plasmid is digested with a different unique restriction enzyme downstream of the gene (e.g., EcoR0109I). The resulting linear fragments are combined, heated to 94° C. to denature the DNA strands and cooled back to room temperature. Heteroduplex DNA under these conditions circularizes and homoduplex DNA remains linear. Thereafter, heteroduplex DNA is easily distinguishable from homoduplex DNA and can be physically separated by agarose gel electrophoresis and cutting out the circular heteroduplex band from the agarose gel. This purified heteroduplex plasmid DNA may be subjected to a nick translation protocol and, after purification, directly transformed into a suitable expression host.

Results achieved using this embodiment with M3 and M4 mutants, SEQ ID Nos: 3 and 4, respectively, were as follows: in XL-1 blue cells, the nick translation reaction resulted in 27 blue colonies out of 212 total colonies, for a 12.74% transformation efficiency; while the heteroduplex control resulted in 18 blue colonies out of 364 total colonies, for a 4.95% transformation efficiency; in XL mutS cells, the nick translation reaction resulted in 55 blue colonies out of 385 total colonies, for a 14.29% transformation efficiency; while the heteroduplex control resulted in 38 blue colonies out of 510 total colonies, for a 7.45% transformation efficiency.

The subsequently described steps, preferably, are carried out with a DNA fragment that has been purified, preferably, by gel electrophoresis, although the reaction can also be carried out on plasmid or phage vector DNA sequences.

Following heteroduplex formation with the variant homologous parental sequences, the heteroduplexes are contacted with a mixture comprising a heteroduplex repair system consisting essentially of a nuclease and a polymerase or, in the instance that a nicked heteroduplex is present, a polymerase. The reaction carried out by a nuclease and polymerase, particularly with DNaseI and Pol I is akin to that of nick translation and is, thus, referred to as such throughout the specification.

The enzymes behind this reaction, a polymerase and a nuclease, or alternatively, the enzyme (polymerase) when the resultant heteroduplex molecule is nicked, are noted as crucial to this process, specifically by carrying out the transcription necessary to resolve mismatches between the variant heteroduplex molecule strands and form a unique double-stranded homoduplex molecule. Accordingly, the combination of these molecules (the polymerase and nuclease) or, in the alternative, the polymerase (when the heteroduplex is nicked) is referred to as a heteroduplex repair system.

While the term “heteroduplex repair system” was coined specifically in recognition of the respective actions of a nuclease and a polymerase on heteroduplex molecules or, alternatively, the action of a polymerase on a nicked heteroduplex molecule, this term is specifically meant to include all other additions to these enzymes which facilitate mismatch repair but do not impact the essential purpose of the two enzymes (i.e., the polymerase and the nuclease) in this invention. In other words, the term “heteroduplex repair system” as used within the instant disclosure refers to any combination of proteins or enzymes wherein the polymerase and polymerase/nuclease combination are considered to be primarily responsible for the heteroduplex repair activity discussed above. This definition, therefore, speaks only to situations wherein another enzyme or protein is not considered an essential contributing factor to the observed activity.

Polymerases of use are known in the art. Preferably, the polymerase is selected from the following: E. coli DNA polymerase, Klenow fragment; reverse transcriptase; T4 DNA polymerase; Native T7 DNA polymerase; chemically modified 17 DNA polymerase; genetically modified T7 DNA polymerase (Δ28); Pfu DNA polymerase; KlenTaq (Ab Peptides™) DNA polymerase; and Taq DNA polymerase. Mutants of DNA polymerases, for instance, mutants of DNA polymerase I are also useful in the instant invention. Accordingly, Pol A5 is also useful in the instant invention.

Most preferably, the enzyme is DNA Pol I. With regard to the polymerase, the time of the reaction is important. Nucleotide replacement along the gene must be kept to a minimum so that read-through by the polymerase on one strand does not negate read-through by other polymerase molecules on the other strand. As one of skill in the art will appreciate, the reaction time can be adapted for optimal results and is related to the concentration of enzymes and reagents in the nick translation reaction. Most preferably, the nick translation reaction is run for a time between 5 minutes and 2 hours.

DNase I, or any other nuclease (be it an endonuclease or an exonuclease) capable of hydrolyzing a double-stranded DNA molecule (e.g., a restriction enzyme), is the other crucial part of the nick translation process, where the molecule has not already been nicked. Preferably, the enzyme used is DNaseI, more preferably DNaseI in the presence of Mg²⁺, which randomly nicks duplex DNA. The amount of DNaseI (or the specific nuclease utilized) can be optimized to the specific application. For example, nick translations of short DNA fragments (<500 basepairs) may require greater concentrations of DNaseI to ensure all molecules are nicked at least once. Preferably, a stock solution is prepared of approximately 1-3 μL DNaseI/20-300 μL 1×NT buffer in 50% glycerol is preferred. Varying amounts of 1-10 μL of this stock can be used, for instance, in a nick translation reaction of approximately 125 μL. Most preferably, the DNaseI is presented in the form of a DNaseI stock which is 3 μL of DNaseI (Stratagene 100,000 U/mL) diluted with 169 μL of 1×NT buffer in 50% glycerol. As one of ordinary skill in the art will appreciate, however, this is all dependent on the specific reaction being carried out. The goal is to nick the heteroduplex DNA in an amount sufficient for the polymerase to enter and mend the DNA mismatches. Caution must be taken, however, not to degrade the DNA. Optimization parameters are readily understood and mastered in the art and are further discussed in e.g., Current Protocols in Molecular Biology (Ausubel et al., eds., 1997).

It is important to note that similar end results to that described above can be achieved with nicked heteroduplex DNA (instead of a nuclease and heteroduplex DNA) and a polymerase. The nicks present in the heteroduplex DNA in this instance accommodate the absence of the nuclease.

Preferred embodiments of the above invention, further, employ DNA ligase in order to seal nicks present in the heteroduplex molecule. This can be effected by treatment with DNA ligase under standard ligating conditions. In particularly preferred embodiments, the DNA ligase employed is T4 DNA ligase. Ligase can be added prior to, during or after the nick translation reaction.

The reaction conditions (e.g., the volume of reaction, concentration of the 4 dNTPs, and amount of DNA) can be varied as will be appreciated by one of ordinary skill in the art; see, e.g., Current Protocols in Molecular Biology, supra. For example, one of ordinary skill in the art is aware that a nick translation reaction can be carried out with as little as 20 ng of DNA and can be scaled down to volumes as small as 5 μL; Id. Moreover, concentrations of dNTPs as low as 2 μM is sufficient for E. coli DNA Polymerase I, although, the polymerase is more efficient when supplied with higher concentrations of substrates; Id.

The temperature of the reaction, as one of ordinary skill in the art will appreciate, is adjusted according to the particular reaction substrates, enzymes, and conditions employed.

Upon completion of the nick translation reaction, the reaction products are purified, digested with unique restriction enzymes and inserted into an appropriate plasmid vector. The plasmid vector with insert is then inserted into a suitable host for expression. The host cells are then screened to identify clones containing the desired mutations.

One of ordinary skill in the art is aware that any of a variety of expression vectors can be used to effect expression of the recombinant polynucleotide. Particularly preferred expression vectors include pUC18 and its derivatives, pUC19 and its derivatives, pBR322 and its derivatives, the pBluescript series, the pGEM series (Promega™), pET series (Promega™), and pESP-1 (Stratagene™). Generally, the specific choice of vector will depend upon the cell type used, the level of expression desired, and the like.

Host cells may be prokaryotic or eukaryotic, including but not limited to, bacteria such as E. coli, fungal cells such as yeast, mammalian cells including, but not limited to, cell lines of human, bovine, porcine, monkey and rodent origin, and insect cells including but not limited to Drosophila and silkworm derived cell lines. Cells and cell lines of particular interest are derived from E. coli K12, E. coli B, Bacillus, and Streptomyces.

Preferably, the screening and selection process is mediated by various markers known in the art, e.g., through luciferase, β-galactosidase, and green fluorescent protein, although any method which would detect a novel activity, function or quality of the recombinant polynucleotide is suitable. Any screening and selection are designed according to what is specifically sought in the recombinant polynucleotide. For instance, an enzyme possessing a particular function or level of function can be tested by measuring or identifying the actual function (or an indicator thereof) of the recombinant protein. One of skill in the art can accommodate a search for any of a range of features into the particular screening. For instance, nucleotide sequences capable of binding to a specific protein can be sought with the labeled protein, or a specific characteristic or quality of the expressed product can be tested according to the features of the quality sought. The use of the instant invention for generating recombinant sequences with enhanced features relative to the parental sequences employed is the most significant use described herein.

A further embodiment wherein the methods of the instant invention can be used is for enhancing directed evolution by PCR mutagenesis and other processes (e.g., error-prone PCR mutagenesis) designed to accrue sequences possessing variations with respect to a particular target sequence. For instance, mutants bearing different beneficial point mutations discovered in one round of directed evolution can be shuffled to generate a library of sequences where one or more of the library members contain collections of mutations onto single genes. These mutants are identified by screening for the desired beneficial activity. This cuts down on both the time and effort required to, one, collect mutations onto one gene using sequence information and site-specific PCR and, two, to gather multiple mutations by performing additional rounds of PCR mutagenesis and screening.

This enhancement is also applicable to random methods of generating novel polynucleotide sequences.

The following non-limiting Examples are presented to better illustrate the invention.

### EXAMPLE 1

Preparation of PCR Products as Substrates for Heteroduplex DNA Reaction

Two PCR reactions were performed to amplify both the M1 and M2 mutants of the Lac Zα gene. The reactions were carried out in the presence of 601.6 μL H₂O; 80 μL 2 mM dNTPs; 80 μL 10×PC 2 Buffer (Ab PEPTIDES™) consisting of 5 mM Tris-HCl pH 9.1, 16 mM ammonium sulfate, 3.5 mM MgCl₂, and 150 μg/mL BSA; 16 μL pUC18 F1 (40 μM): (5′-TGAGCGTCGTTTTTGTGAT; SEQ ID NO:6); 16 μL pUC18 R1 (40 μM): (5′-TAGGGGTTCCGCGCACATTT; SEQ ID NO:7); 2.4 μL POL MIX consisting of 15:1 Klentaq (Ab PEPTIDES™): PFU Polymerase (STRATAGENE™); and 4 μL Template DNA (PROMEGA™ miniprep of pUC18 plasmid M1 or M2 (M1:SEQ ID NO:1; M2:SEQ ID NO:2) −200 ng/μL). A total volume of 800 μL was divided into eight 0.6 mL PCR tubes and run on a thermal cycler using the following program: 94° C. for 2 minutes; 25 cycles of [94° C. for 1 minute; 52° C. for 1 minute; 72° C. for 1 minute, 10 seconds], 72° C. for 7 minutes; and 6° C. until purified.

PCR Products were purified using PROMEGA™ PCR Preps Kit with Wizard Minicolumns. 200 μL of PCR reaction was purified per minicolumn and the product was eluted with 50 μL of H₂O. A total volume of 200 μL was generated for both M1 and M2 pUC18 F1R1 PCR products.

A₂₆₀ of PCR products was measured using a Spectra Max Plus spectrophotometer (MOLECULAR DEVICES™) to determine concentration of DNA. The results were as follows: M1 pUC18 F1R1=175 ng/μL; and M2 pUC18 F1R1=208.75 ng/μL.

### EXAMPLE 2

Heteroduplex DNA Formation

170 μL of M1 pUC18F1R1; 143 μL of M2 pUC18F1R1; and 16.47 μL of 20×TrisKCl heteroduplex buffer (0.3 M tris pH 7.83, 1.2 M KCl) were mixed and the contents divided into 3×0.6 mL PCR tubes. The tubes were then set on a thermal cycler for the following heteroduplex program:

94° C. for 4 minutes; ramp to 90° C. and hold for 10 minutes; ramp to 85° C. and hold for 10 minutes; ramp to 80° C. and hold for 10 minutes; continue this ramp down in temperature at 5° C. increments and hold 10 minutes each until 60° C. is reached and then ramp to 40° C. and hold 10 minutes; then 4° C. until reaction is purified.

Note that all temperature ramps are performed at 0.1° C./sec. Heteroduplex DNA was purified using three Promega Wizard columns with the PCR prep Kit, and DNA was eluted in H₂O for a combined volume of 150 μL of heteroduplex DNA.

### EXAMPLE 3

Nick Translation Reaction

The following reagents were mixed on ice: 40 μL of pUC 18 F1R1 purified heteroduplex DNA; 40 μL of H₂O; 12.5 μL of 10×NT buffer (0.5M Tris HCl pH 7.5; 0.1M MgCl₂; 10 mM Dithiothreitol (DTT); 0.5 mg/mL BSA); 5 μL of DNA Pol I (New England Biolabs); 22.5 μL of 2 mM dNTPs; and 5 μL of 1× DNaseI stock (dilute 3 μL of DNaseI (Stratagene, 100,000 U/mL) with 169 μL of 1×NT buffer in 50% glycerol).

The nick translation reaction was run at 14° C. for 15 minutes. The reaction was stopped with addition of 2 μL of 0.5 M EDTA pH 8.0. The nick translation reaction was purified using Promega PCR preps kit and DNA was eluted with 60 μL of H₂O.

### EXAMPLE 4

Restriction Digest of Nick Translation DNA and Heteroduplex Control

The following reagents were combined: 52 μL of nick translation DNA; 7 μL of NEB 4 buffer (NEB; New England Biolabs™); 0.7 μL 10×BSA (NEB); 1 μL of EcoRI (NEB); 4 μL of Eco 0109I (NEB); 4 μL Sap I (NEB); and 2 μL of H₂O.

A heteroduplex control was prepared by combining the following reagents: 25 μL of pUC 18 F1R1 heteroduplex DNA (same stock as used in nick translation reaction); 7 μL of NEB 4 buffer (NEB); 0.7 μL 10×BSA (NEB); 1 μL of EcoRI (NEB); 4 μL of Eco 01091 (NEB); 4 μL Sap I (NEB); and 28 μL of H₂O.

The rest of the reactions were carried out with both the nick translation DNA and the heteroduplex control separately.

Restriction reactions were run at 37° C. for 5 hours.

Nick translation and control Lac Zα insert bands were isolated from 0.8% agarose gel using a razor blade. The gel fragment was purified with Promega PCR Preps kit and the inserts were eluted with 50 μL of H₂O.

### EXAMPLE 5

Ligation Reaction

The following reagents were combined: 0.5 μL pUC18 vector DNA with Lac Zα insert removed with EcoRI and Eco 0109I; 5 μL of F1R1 control insert DNA or 3 μL of nick translation insert DNA; 1 μL of 10×Ligation Buffer (Boerhinger Manneheim); 0.75 μL of T4 DNA Ligase (Boerhinger Manneheim); and 2.75 μL of H₂O for control or 4.75 μL of H₂O for nick translation control.

Ligation reactions were run at 16° C. overnight.

### EXAMPLE 6

Transformation Reaction

XL-1 Blue competent cells (Stratagene) were defrosted and 50 μL was aliquotted per 0.6 mL PCR tube. 5 μL of ligation reaction was added to each tube and 0.75 μL of pUC 18 (Stratagene) plasmid was added as a control. The DNA was incubated with E coli on ice for 30 minutes. The cell/DNA mixture was then put on a PCR block for 1 minute, 30 seconds, at 42° C. The heat shocked cell/DNA mixture was then immediately placed on ice for 2 minutes. 450 μL of S.O.C. (Gibco BRL)/tube was then added and the tube contents were then transferred to 15 mL tubes (Falcon) and shaken at 37° C. for 1 hour. Two 250 μL aliquots of each transformation and one 30 μL aliquot of the wildtype pUC 18 control were then plated onto LB Agar Ampicillin and X-Gal and placed at 37° C. overnight.

### EXAMPLE 7

Results

The number of colonies on half the plate were counted and multiplied by two to approximate the number of colonies per plate. The total number of blue colonies on each plate were counted. For the pUC 18 control plate, all colonies were counted. The results were as follows: the nick translation reaction resulted in 5 blue colonies out of 524 total colonies, for a 0.95% reversion frequency; the heteroduplex control resulted in 7 blue colonies out of 1,032 total colonies, for a 0.54% reversion frequency; and the pUC 18 control resulted in 45 blue colonies out of 47 total colonies, or 95.74% blue colonies.

### EXAMPLE 8

Nick Translation on two pUC 18 Mutants Lacking any Insertions or Deletions of Bases Relative to the Wildtype Lac Zα Genome.

Two pUC 18 mutants of Lac Z alpha M3 and M4 (M3:SEQ ID NO: 3; M4; SEQ ID NO:4) were constructed without any insertion or deletion of bases relative to the wildtype genome. Heteroduplex DNA was generated in accordance with the method described in Arnold et al., WO 99/29902 (see also, Westmoreland et al., 1997 Genetics 145:29-38; which are both hereby incorporated by reference.

Briefly, one mutant plasmid was digested with a unique restriction enzyme (EcoRI) upstream of the gene and the other mutant was digested with a different unique restriction enzyme (Eco0109I) downstream of the gene. The linear fragments were combined, heated to 94° C. to denature the DNA strands and cooled back to room temperature. Heteroduplex DNA (one strand nicked with EcoRI, and the second strand nicked by Eco0109I) circularized and homoduplex DNA (both strands cut with either EcoRI or Eco0109I) remained linear. In this manner, heteroduplex easily distinguishable from the homoduplex DNA can be physically separated from the linear homoduplex by gel electrophoresis and by cutting out the heteroduplex band from the agarose gel. The heteroduplex DNA can then be purified using Promega™ PCR Preps kit.

The above experiments were carried out to generate heteroduplex plasmid DNA. Some of this heteroduplex plasmid DNA was contacted with nick translation enzymes as in Example 3 above. A portion of the heteroduplex plasmid was not contacted with nick translation enzymes such that it could be used as the heteroduplex negative control. The two plasmid preparations were transformed separately into both XL-1 Blue and XL mutS (a strain with the mutS gene knocked out). The results in the mutS cells were slightly better than in XL-1 blue cells suggesting that the mutHLS system is not involved in the recombination. In these experiments the total number of blue and white colonies were counted in a defined area on the agar plates.

The results were as follows: in XL-1 blue cells, the nick translation reaction resulted in 125 blue colonies out of 1,031 total colonies, for a 12.12% reversion frequency; while the heteroduplex control resulted in 82 blue colonies out of 1,315 total colonies, for a 6.24% reversion frequency; in XL mutS cells, the nick translation reaction resulted in 55 blue colonies out of 385 total colonies, for a 14.29% reversion frequency; while the heteroduplex control resulted in 38 blue colonies out of 510 total colonies, for a 7.45% reversion frequency.

### EXAMPLE 9

Nick Translation (and Treatment with Pol I only) on M3 and M4 Mutants

Two pUC 18 mutants of Lac Z alpha M3 and M4 (M3:SEQ ID NO: 3; M4:SEQ ID NO:4) were constructed without any insertion or deletion of bases relative to the wildtype genome. Heteroduplex DNA was generated in accordance with the method described in Arnold et al., WO 99/29902; which is hereby incorporated by reference.

Briefly, one mutant plasmid was digested with a unique restriction enzyme upstream of the gene and the other mutant was digested with a different unique restriction enzyme downstream of the gene. The linear fragments were combined, heated to 94° C. to denature the DNA strands and cooled back to room temperature. Heteroduplex DNA circularized and homoduplex DNA remained linear. In this manner, heteroduplex easily distinguishable from the homoduplex DNA can be physically separated from the linear homoduplex by gel electrophoresis and by cutting out the heteroduplex band from the agarose gel. The heteroduplex DNA can then be purified using Promega™ PCR Preps kit.

The above experiments were carried out with both the heteroduplex negative control, the heteroduplex treated with Pol I only, and the heteroduplex plasmid treated with the nick translation enzymes (both Pol I and DNase I). Then the plasmid preparations were transformed into XL-1 Blue. In this experiment, you will note that the heteroduplex DNA treated with Pol I only shows an improved frequency of recombination over that of the heteroduplex control. The nick translation-treated heteroduplex molecules still fare the best. In these experiments the total number of blue and white colonies were counted in a defined area on the agar plates.

The results were as follows: the nick translation reaction resulted in 46 blue colonies out of 390 total colonies, for a 11.79% reversion frequency; the Pol I—only treated heteroduplex resulted in 78 blue colonies out of 829, for a 9.41% reversion frequency; and the heteroduplex control resulted in 46 blue colonies out of 626 total colonies, for a 7.35% reversion frequency.

What is claimed:

1. A method for producing recombinant polynucleotides, which comprises:
(a) providing variant homologous parental sequences;
(b) incubating said sequences under conditions which promote heteroduplex formation; and
(c) contacting said heteroduplexes with a mixture comprising a heteroduplex repair system consisting essentially of a nuclease and a polymerase.

2. A method in accordance with claim 1 further comprising:
(d) identifying the recombinant polynucleotides produced.

3. A method in accordance with claim 1 wherein the heteroduplexes are contacted with a mixture comprising a heteroduplex repair system consisting of a nuclease and a polymerase.

4. A method in accordance with claim 1 wherein the nuclease is an endonuclease.

5. A method in accordance with claim 4 wherein the endonuclease is DNase I.

6. A method in accordance with claim 1 wherein the nuclease is a restriction enzyme.

7. A method in accordance with claim 1 wherein the nuclease is an exonuclease.

8. A method in accordance with claim 1 wherein the polymerase is DNA polymerase I.

9. A method in accordance with claim 1 wherein the nuclease is DNase I and the polymerase is DNA polymerase I.

10. A method in accordance with claim 1 wherein the nuclease is a restriction enzyme and the polymerase is DNA polymerase I.

11. A method in accordance with claim 1 wherein the homologous sequences are derived from two or more different genes.

12. A method in accordance with claim 1 wherein the homologous sequences are derived from the same gene.

13. A method in accordance with claim 12 wherein at least one of the homologous sequences is a mutant of Lac Za.

14. A method in accordance with claim 1 wherein the homologous sequences are on plasmids.

15. A method in accordance with claim 14 wherein the method further comprises linearizing the plasmids prior to step (b).

16. A method in accordance with claim 1 wherein the mixture further comprises DNA ligase.

17. A method in accordance with claim 16 wherein the ligase is T4 DNA ligase.

18. A method in accordance with claim 1 wherein the homologous parental sequences are DNA.

19. A method in accordance with claim 1 wherein heteroduplex formation is achieved by heating and annealing of the homologous parental sequences.

20. A method in accordance with claim 1 wherein heteroduplex formation is achieved via single strand PCR of complementary strands of the homologous parental sequences which are subsequently annealed.

21. A method in accordance with claim 20 wherein the single strand PCR is carried out with more than one primer.

22. A method in accordance with claim 21 wherein the PCR is carried out with primers comprising a forward primer for one strand of a parental sequence and a reverse primer for the complementary sequence on another parental sequence.

23. A method in accordance with claim 1 wherein heteroduplex formation involves amplifying the homologous parental sequences.

24. A method in accordance with claim 23 wherein heteroduplex formation involves amplifying single stranded plasmid DNA homologous parental sequences.

25. A method in accordance with claim 23 wherein the amplification is carried out by M13-derived vectors and helper phage.

26. A method in accordance with claim 1 wherein step (d) comprises
(a) purifying a polynucleotide product of step (c);
(b) digesting said polynucleotide product for insertion into a vector;
(c) ligating the digested polynucleotide product into a vector;
(d) expressing said vector in a suitable host; and
(e) identifying polynucleotide clones exhibiting a novel characteristic or function with respect to the parental sequences; said novel polynucleotide clones comprising the recombinant polynucleotides.

27. A method in accordance with claim 26 wherein the method further comprises isolating the recombinant polynucleotides from the novel polynucleotide clones.

28. A method for producing recombinant polynucleotides, which comprises:
(a) providing variant homologous parental sequences;
(b) incubating said sequences under conditions which promote heteroduplex formation wherein the resultant heteroduplexes are nicked; and
(c) contacting said heteroduplexes with a mixture comprising a heteroduplex repair system consisting essentially of a polymerase.

29. A method in accordance with claim 28 further comprising:
(d) identifying the recombinant polynucleotides produced.

30. A method in accordance with claim 28 wherein the mixture consists of a polymerase.

31. A method in accordance with claim 28 wherein the polymerase is DNA polymerase I.

32. A method in accordance with claim 28 wherein the homologous parental sequences are DNA.

33. A method in accordance with claim 28 wherein step (d) comprises
(a) purifying a polynucleotide product of step (c);
(b) digesting said polynucleotide product for insertion into a vector;
(c) ligating the digested polynucleotide product into a vector;
(d) expressing said vector in a suitable host; and
(e) identifying polynucleotide clones exhibiting a novel characteristic or function with respect to the parental sequences; said novel polynucleotide clones comprising the recombinant polynucleotides.

34. A method in accordance with claim 33 wherein the method further comprises isolating the recombinant polynucleotides from the novel polynucleotide clones.

35. A method of repairing mismatched nucleic acid molecules in vitro which comprises contacting the mismatched molecule with a mixture consisting essentially of a polymerase and a nuclease.

36. A method in accordance with claim 35 wherein the mixtures consists of a polymerase and a nuclease.

37. A method in accordance with claim 35 wherein the nuclease is an endonuclease.

38. A method in accordance with claim 37 wherein the nuclease is DNase I.

39. A method in accordance with claim 37 wherein the nuclease is a restriction enzyme.

40. A method in accordance with claim 35 wherein the nuclease is an exonuclease.

41. A method in accordance with claim 35 wherein the polymerase is DNA polymerase I.

42. A method in accordance with claim 35 wherein the nuclease is DNase I and the polymerase is DNA polymerase I.

43. A method in accordance with claim 35 wherein the nuclease is a restriction enzyme and the polymerase is DNA polymerase I.

44. A method in accordance with claim 35 wherein the homologous parental sequences are DNA.

45. A method of repairing mismatched nucleic acid molecules in vitro wherein the mismatched molecule is nicked which comprises contacting the mismatched heteroduplexes with a mixture consisting essentially of a polymerase.

46. A method in accordance with claim 45 wherein the mixture consists of a polymerase.

47. A method in accordance with claim 45 wherein the polymerase is DNA polymerase I.

48. A method in accordance with claim 45 wherein the homologous parental sequences are DNA.


SEQUENCE LISTING (9 sequences)

SEQ ID NO: 1 - DNA, 315 bp, Escherichia coli
atgattacgaattcgagctcggtacccggggatcctctagagtcgacctcgagccatggctaactaattaagtaatttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatag

SEQ ID NO: 2 - DNA, 321 bp, Escherichia coli
atgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagttaactaattaactaagatatcgcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatag

SEQ ID NO: 3 - DNA, 318 bp, Escherichia coli
atgattacgaattcgagctcggtacccggggatcctctagagtcgacctgcaggcatgcaagctaactaattaagtaagttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatag

SEQ ID NO: 4 - DNA, 318 bp, Escherichia coli
atgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctaactaattaactaagtggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatag

SEQ ID NO: 5 - DNA, 318 bp, Escherichia coli
atgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatag

SEQ ID NO: 6 - DNA, 19 bp, Artificial Sequence
  PCR primer
tgagcgtcgtttttgtgat

SEQ ID NO: 7 - DNA, 20 bp, Artificial Sequence
  PCR primer
taggggttccgcgcacattt

SEQ ID NO: 8 - DNA, 318 bp, Artificial Sequence; identical to SEQ ID NO: 5
  Consensus sequence

SEQ ID NO: 9 - DNA, 318 bp, Artificial Sequence; identical to SEQ ID NO: 5
  Consensus sequence